(19) United States
(12) Patent Application Publication
(10) Pub. No.: US 2023/0015281 A1
(43) Pub. Date: Jan. 19, 2023

(54) ALIGNMENT TOOL FOR ALIGNING HEART VALVE WITH DELIVERY SYSTEM

(71) Applicant: BOSTON SCIENTIFIC SCIMED, INC., MAPLE GROVE, MN (US)

(72) Inventors: Tim O'Connor, Galway (IE); Enda Hannon, Galway (IE); John Lardner, Galway (IE); Declan Loughnane, Galway (IE); Pearse A. Coffey, Galway (IE)

(73) Assignee: BOSTON SCIENTIFIC SCIMED, INC., MAPLE GROVE, MN (US)

(21) Appl. No.: 17/861,555

(22) Filed: Jul. 11, 2022

Related U.S. Application Data
(60) Provisional application No. 63/221,118, filed on Jul. 13, 2021.

(51) Int. Cl. A61F 2/95; G02B 25/00

(52) U.S. Cl. CPC A61F 2/95; G02B 25/005; A61F 2002/9505; A61F 2002/9534

(57) ABSTRACT

An alignment tool for loading a stent includes a plurality of arms each having a shaft with an engagement region moveable between a first, angled configuration relative to the shaft, and a second, straight configuration, with each engagement region having an inner surface shaped to mate with a stent holder. The alignment tool further includes a lock ring having a lumen configured to receive the plurality of arms, with the lock ring configured to slide over the arms between a first retracted position in which the engagement region of each arm is exposed and allowed to bias into the angled configuration, and a second locked position in which the lock ring extends over at least a portion of the engagement regions and compresses the engagement regions into the straight configuration. The alignment tool may also include a spring configured to bias the lock ring in the locked position.

### CROSS-REFERENCE TO RELATED APPLICATIONS

This application claims the benefit of priority of U.S. Provisional Application No. 63/221,118 filed Jul. 13, 2021, the entire disclosure of which is hereby incorporated by reference.

### TECHNICAL FIELD

The disclosure pertains to medical devices and more particularly to devices for aligning a heart valve during loading into a delivery system, and methods for using such medical devices.

### BACKGROUND

A wide variety of medical devices have been developed for medical use including, for example, artificial heart valves for repair or replacement of diseased heart valves. The artificial heart valve must be aligned precisely as it is loaded into a delivery system. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example alignment tool for loading a stent includes a plurality of arms each having a shaft with a first end including an engagement region and a second opposite end, wherein each engagement region is moveable between a first, angled configuration relative to the shaft, and a second, straight configuration, a lock ring having a lumen configured to receive the plurality of arms, the lock ring configured to slide over the arms between a first retracted position in which the engagement region of each arm is exposed and allowed to bias into the angled configuration, and a second locked position in which the lock ring extends over at least a portion of the engagement regions and compresses the engagement regions into the straight configuration, and a spring configured to bias the lock ring in the locked position.

Alternatively or additionally to the embodiment above, each engagement region has a longitudinal slit extending from a free end of the engagement region towards the second end of the arm, the longitudinal slit allowing the engagement region to expand when in the angled configuration.

Alternatively or additionally to any of the embodiments above, each arm includes a cut-out region in a side surface of the engagement region, wherein the cut-out regions in adjacent arms form an opening when the adjacent arms are in the straight configuration, the opening configured to receive a stent loop.

Alternatively or additionally to any of the embodiments above, the lock ring includes at least one magnifying lens configured to be positioned over the opening.

Alternatively or additionally to any of the embodiments above, the lock ring is transparent.

Alternatively or additionally to any of the embodiments above, each arm has an enlarged portion at the second end of the shaft, wherein the enlarged portion extends radially outward farther than a diameter of the spring.

Alternatively or additionally to any of the embodiments above, each arm has a raised transverse rib spaced apart from the enlarged portion.

Alternatively or additionally to any of the embodiments above, the lock ring includes a rear shoulder extending into the lumen, the rear shoulder configured to engage the transverse rib when the lock ring is in the second locked position.

Alternatively or additionally to any of the embodiments above, the lock ring includes a front shoulder extending into the lumen, the front shoulder configured to slide along the arms and move the engagement regions of the arms from the angled configuration to the straight configuration.

Alternatively or additionally to any of the embodiments above, each of the plurality of arms is identical.

Alternatively or additionally to any of the embodiments above, the lock ring has a front end disposed adjacent the engagement region of the arms, and an opposite rear end, wherein the lock ring has an enlarged flared rear end with a diameter larger than a diameter of the front end.

Alternatively or additionally to any of the embodiments above, each engagement region includes an inner surface shaped to mate with a stent holder.

Alternatively or additionally to any of the embodiments above, at least at the second end of the plurality of arms, side edges of each arm abut side edges of adjacent arms, thereby defining a channel extending through the plurality of arms.

Alternatively or additionally to any of the embodiments above, the side edges of each arm abut side edges of adjacent arms along a length of each shaft in all regions but the engagement region of each arm.

Another example alignment tool for loading a stent onto a stent holder includes a plurality of arms each having a shaft with a first end portion including an engagement region and an enlarged second end portion, wherein each engagement region is moveable between a first, angled configuration relative to the shaft, and a second, straight configuration, wherein side edges of each arm in at least the enlarged second end portion abut side edges of adjacent arms to define a channel a lock ring having a lumen configured to receive the shaft of the plurality of arms, the lock ring configured to slide over the arms between a first retracted position in which the engagement region of each arm is exposed and allowed to bias into the angled configuration, and a second locked position in which the lock ring extends over at least a portion of the engagement regions and compresses the engagement regions into the straight configuration, and a spring configured to bias the lock ring in the locked position.

Alternatively or additionally to the embodiments above, each arm includes a cut-out region in a side surface of the engagement region, wherein the cut-out regions in adjacent arms form an opening when the adjacent arms are in the straight configuration, the opening configured to receive a stent loop.

Alternatively or additionally to any of the embodiments above, each arm has a raised transverse rib spaced apart from the enlarged portion, the raised transverse rib on all of the arms collectively forming a raised ring.

Alternatively or additionally to any of the embodiments above, the lock ring includes a rear shoulder extending into the lumen, the rear shoulder configured to engage the raised ring when the lock ring is in the second locked position.

Alternatively or additionally to any of the embodiments above, the lock ring includes a front shoulder extending into the lumen, the front shoulder configured to slide along the arms and move the engagement regions of the arms from the angled configuration to the straight configuration.

An example method of loading a stent onto a stent holder using an alignment tool includes inserting the stent having a plurality of terminal end loops into the stent holder having a plurality of pins on which the terminal end loops are to be placed, placing the alignment tool over the stent holder, the alignment tool having a plurality of arms each having a shaft with a first end including an engagement region and a second opposite end, wherein each engagement region is moveable between a first, angled configuration relative to the shaft, and a second, straight configuration, each engagement region having an inner surface shaped to mate with the stent holder, wherein each arm includes a cut-out region in a side surface of the engagement region, wherein the cut-out regions in adjacent arms form an opening configured to receive a stent loop and a pin, a lock ring having a lumen configured to receive the plurality of arms, the lock ring configured to slide over the arms between a first retracted position in which the engagement region of each arm is exposed and allowed to bias into the angled configuration forming a loading zone for receiving the stent holder, and a second locked position in which the lock ring extends over at least a portion of the engagement regions and compresses the engagement regions into the straight configuration to secure the stent holder, and a spring configured to bias the lock ring in the locked position, wherein the alignment tool is placed over the stent holder with the lock ring in the first retracted position such that the pins on the stent holder are received within the opening in the engagement region. The method further includes releasing the lock ring and allowing it to move into the locked position, moving the engagement region of each arm into its straight configuration, advancing the stent and moving one stent loop over each pin, compressing the stent onto the stent holder, moving the lock ring on the alignment tool to the retracted position to release the stent holder, and removing the alignment tool from the stent holder and the stent.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1A illustrates a stent loop positioned adjacent a pin on a stent holder before compression;

FIG. 1B illustrates the stent holder and stent of FIG. 1A with the stent loop correctly aligned over the pin and compressed;

FIG. 1C illustrates the stent holder and stent of FIG. 1A with the stent loop misaligned and compressed next to the pin;

FIG. 2 is a perspective view of an example alignment tool in the open position;

FIG. 3 is a perspective view of the alignment tool of FIG. 2 in the closed position;

FIG. 4 is an exploded view of the alignment tool of FIG. 2;

FIG. 5 is a partial cut away view of the alignment tool of FIG. 2 in the open position;

FIG. 6 is a partial cut away view of the alignment tool of FIG. 2 in the closed position;

FIG. 7 is a partial cut away view of the alignment tool of FIG. 2 in the open position;

FIG. 8 is a partial cut away view of the alignment tool of FIG. 2 in the closed position;

FIG. 9 is a close-up view of a portion of one arm of the alignment tool of FIG. 2; and

FIG. 10 is a perspective view of the alignment tool of FIG. 2 with a stent holder inserted.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Current artificial heart valves, such as the replacement valve and expandable anchor described in U.S. Pat. No. 8,992,608, must be loaded precisely into a delivery catheter, such as those described in U.S. Pat. Nos. 10,245,145 and 10,682,228, the disclosures of which are incorporated herein by reference. The artificial heart valve may include a stent portion with loops that must be compressed and aligned precisely in a delivery catheter just before implantation. The loading step can be complex and difficult and generally occurs in the Catheter Lab. The components, including pins on the stent holder and loops on the stent, are small and difficult to see to achieve precise alignment. The difficulties associated with the alignment steps increase the risk of misloading the valve with negative consequences for the loading time if the misload is identified and/or for the clinical result in terms of non-optimal implant positioning if the misload is not identified. Only one misload is generally permitted, and after a second misload the valve and delivery system must be discarded. The loading of the artificial heart valve and associated stent is a critical part of the implantation procedure, and improvements are desired.

Applicants have developed an automatic alignment tool that facilitates precise alignment of the stent portion of an artificial heart valve with the delivery system to enable smooth preparation and expedite the loading process. Automatic alignment of the valve with the delivery system will help the individual loading the valve, reducing stress and anxiety in a pressurized Catheter Lab environment. In some examples, the heart valve being loaded may be a transcatheter aortic valve replacement (TAVR) such as the ACURATE™ aortic valve system of Boston Scientific.

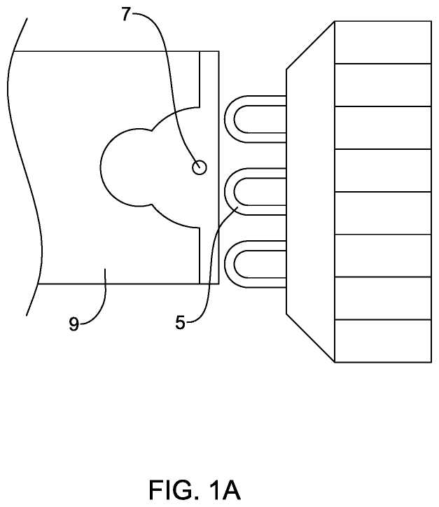

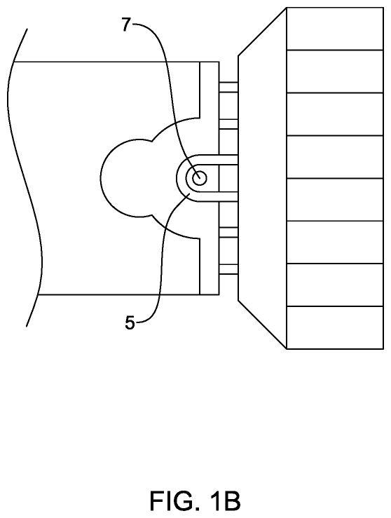

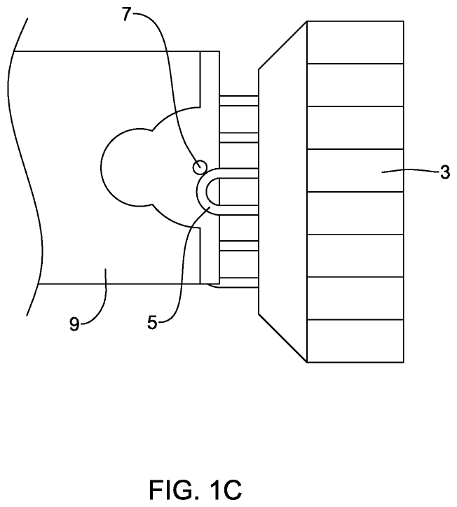

FIGS. 1A-1C illustrate loading the stent portion of a heart valve into a delivery device and some complications that may arise. The loops 5 of the stent portion at the distal end of the valve must be precisely aligned with pins 7 on the stent holder 9 component of the delivery system. FIG. 1A illustrates a loop 5 that must be moved into alignment to engage the pin 7 on the stent holder 9 prior to compression of the stent. The loops and pins are small and difficult to see. For example, the loops may be 1.5 mm and the pins may be 0.5 mm (0.060 inch and 0.020 inch, respectively), approximately the size of a ballpoint pen tip, which makes precise alignment difficult. FIG. 1B illustrates the correct alignment of the loop 5 over the pin 7 and compression of the stent. A complication that can lead to a missed loading of the valve is when one of the three valve loops 5 is mis-aligned with one of the three pins 7 on the stent holder, as shown in FIG. 1C. When the valve is then compressed by the loading tool 3, there is a potential for sheathing the valve on the stent holder 9 without all three loops being correctly engaged with the three pins. Once the device is sheathed, any misalignment may be difficult to see. If this device was deployed in a clinical scenario, the valve positioning and coaxial alignment could be compromised. The onus is on the individual loading the valve to identify any misalignments in a catheter lab and so alignment can be a significant cause of stress and anxiety. Even if the issue is identified the valve loading procedure must be restarted from the beginning leading to a scenario where the physician is waiting for the loaded valve and the TAVR procedure time is extended.

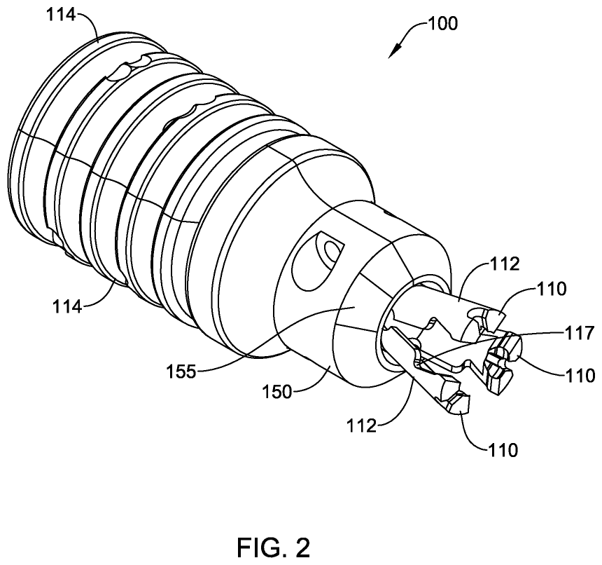

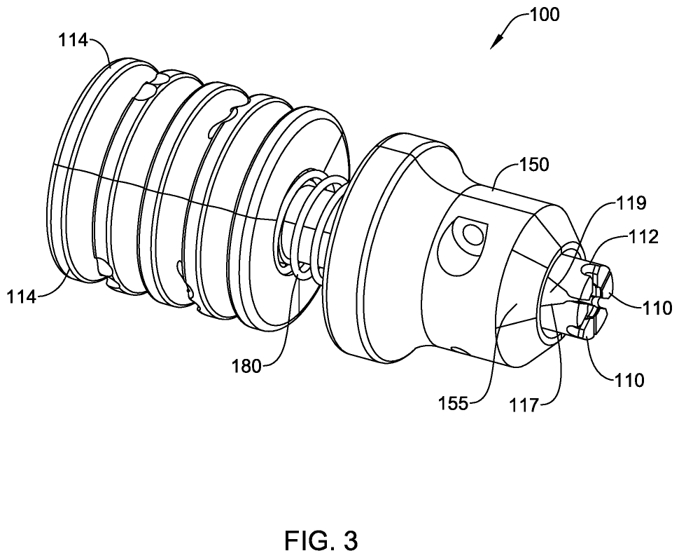

As will be described in greater detail below, FIG. 2 illustrates an example alignment tool 100 including a plurality of arms 110 and a lock ring 150, with the arms 110 in an open position. Each arm 110 may have a first end defining an engagement region 112 and an opposite second end 114. The example shown in FIG. 2 includes three arms 110. The alignment tool 100 in FIG. 2 is shown with the lock ring 150 in the retracted position and the engagement regions 112 of the arms 110 in the biased open configuration. The side surfaces of each arm 110 in the engagement region 112 may include a cut-out region 117 configured to receive a stent loop and pin as described in more detail below. FIG. 3 shows the alignment tool 100 with the arms 110 in the closed position. The lock ring 150 is in the forward locked position and the engagement regions 112 of the arms 110 are in the straight configuration, with the cut-out regions 117 of adjacent arms 110 forming an opening 119. In some examples, the lock ring 150 may be transparent, which may make it easier for the user to view the alignment of the pin and stent loop within the opening 119. In other examples, the lock ring 150 may include at least one magnifying lens 155 configured to be positioned over the opening 119, to aid in aligning the pin and stent loop. In some examples, a magnifying lens 155 may be positioned over each opening 119 to allow for easier viewing of each stent loop and pin. The alignment tool 100 may include a spring 180 configured to bias the lock ring 150 in the forward locked position. In other examples, the lock ring 150 may move along the arms 110 in a friction fit such that it holds whatever position it is moved into.

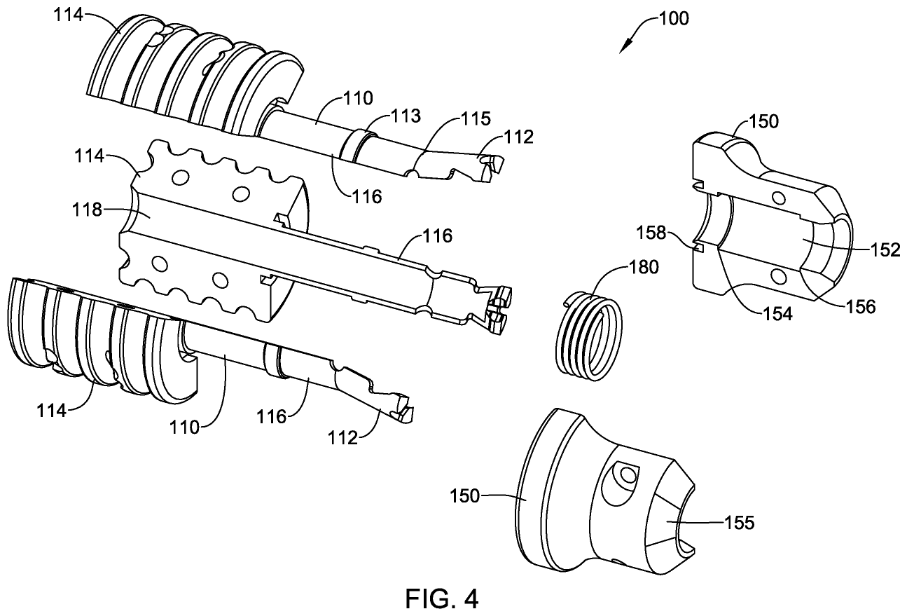

Details of the arms 110 and lock ring 150 are illustrated in the exploded view in FIG. 4. Each arm 110 may have a shaft 116 with a first end including the engagement region 112 and the second opposite end 114. The engagement regions 112 may be moveable between a first, angled configuration relative to the shaft 116, as shown in FIG. 2, and a second, straight configuration as shown in FIG. 3. The engagement region 112 may be joined to the shaft 116 by a flexible hinge 115. The engagement regions 112 may be biased in the angled configuration in which the engagement regions 112 extend radially outward from the longitudinal axis of the shaft 116. The engagement regions 112 may be moved to the straight configuration in which the engagement regions 112 are aligned axially with the shafts 116. Sliding the lock ring 150 over the engagement regions 112 provides sufficient force to move the engagement regions 112 into the straight configuration, as shown in FIG. 3.

Each arm 110 may have an enlarged portion 114 at the second end of the shaft 116, with the enlarged portion 114 extending radially outward farther than a diameter of the spring 180. The enlarged portion 114 of each arm 110 may include ridges or have a textured surface to aid in grasping it during use. Each arm 110 may be a single, monolithic piece. In other examples, the engagement region 112 may be formed separately and joined to the shaft 116 in a biased angled configuration. In some examples, the inner surface of each arm 110 may be curved to form a channel 118 when the arms 110 are positioned adjacent one another. The channel 118 may be sized to receive a portion of the stent holder (not shown). Each arm 110 may also include a raised transverse rib 113 spaced apart from the enlarged portion 114. In some examples, all of the plurality of arms 110 may be identical in structure. When the arms 110 are positioned adjacent one another, the transverse rib 113 on each arm 110 may collectively form a circumferential rib or ring 113. In the example shown in the figures, the alignment tool 100 includes three arms 110.

The lock ring 150 may define a lumen 152 configured to receive the arms 110 in a sliding engagement, as shown in FIGS. 2 and 3. In some examples, the lock ring 150 may be formed in two halves, as shown in FIG. 4, and fixed together over the arms 110. In other examples, the lock ring 150 may be a single, monolithic element. The lock ring 150 may be configured to slide over the arms 110 between a first retracted position (FIG. 2) in which the engagement region 112 of each arm 110 is exposed and allowed to bias into the angled configuration, and a second locked position (FIG. 3) in which the lock ring 150 extends over at least a portion of the engagement regions 112 and compresses the engagement regions 112 into the straight configuration. The lock ring 150 may include a rear shoulder 154 and a front shoulder 156 extending into the lumen 152. In some examples, the lock ring 150 may include a recess 158 configured to receive the spring 180.

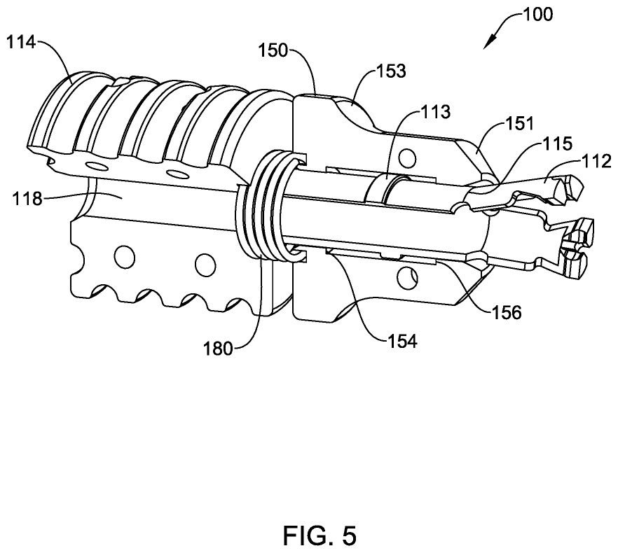

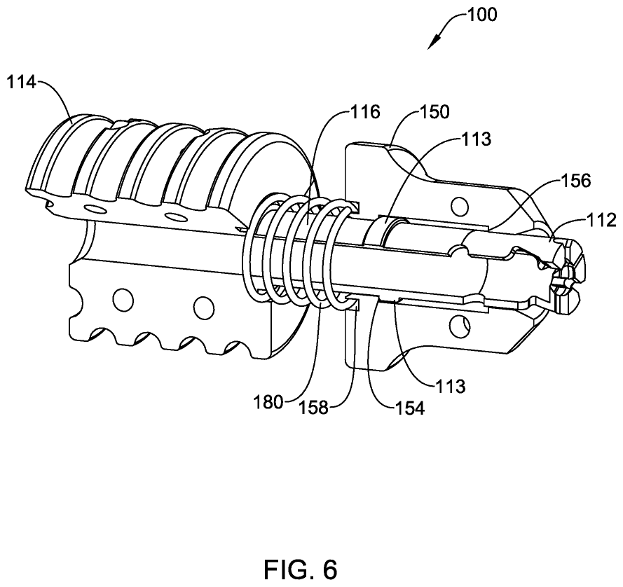

FIGS. 5 and 6 illustrate the sliding movement of the lock ring 150 over the arms 110 to actuate the engagement regions 112 between the open configuration (FIG. 5) and the closed configuration (FIG. 6). In the figures, one arm 110 and half of the lock ring 150 have been removed to show details of the inner structures. The channel 118 defined by inner surfaces of the arms 110 extends completely through the alignment device 100, configured to receive the stent holder (not shown). In the open configuration, the lock ring 150 is pushed toward the enlarged portions 114 of the arms 110, compressing the spring 180 and moving the front end 151 of the lock ring 150 to a position rearward of the hinge 115, allowing the engagement regions 112 to move into the biased angled configuration, as shown in FIG. 5. In some examples, the lock ring 150 may have a tapered or flared rear end 153 to aid in grasping and moving the lock ring 150 rearward into the open configuration. The flared rear end 153 may have a diameter larger than a diameter of the front end 151 of the lock ring 150. Releasing the lock ring 150 allows the spring 180 to expand which pushes the lock ring 150 forward until the rear shoulder 154 engages the rib 113, as shown in FIG. 6. The rib 113 may prevent the lock ring 150 from sliding off the arms 110. As the lock ring 150 moves forward, the front shoulder 156 slides along the arms 110 and pushes the engagement regions 112 downward and into the straight configuration in which the engagement regions 112 are substantially aligned with the shafts 116. The enlarged portion 114 of the arms 110 may form a rear stop for the spring 180. In some examples, the enlarged portion 114 may include a recess to engage a portion of the spring 180.

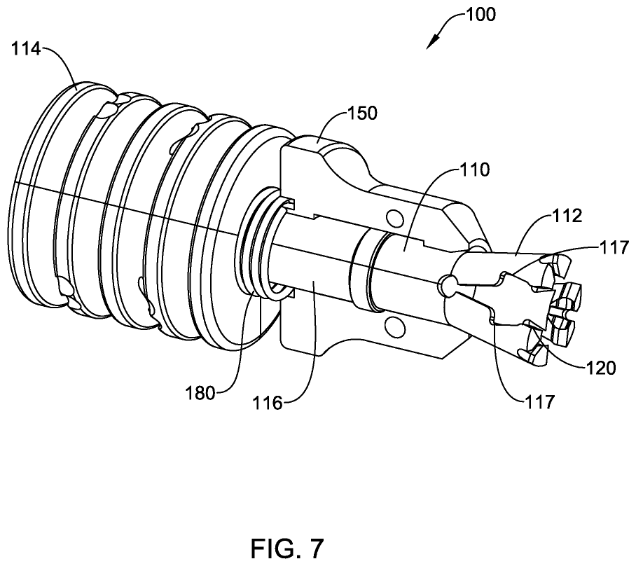

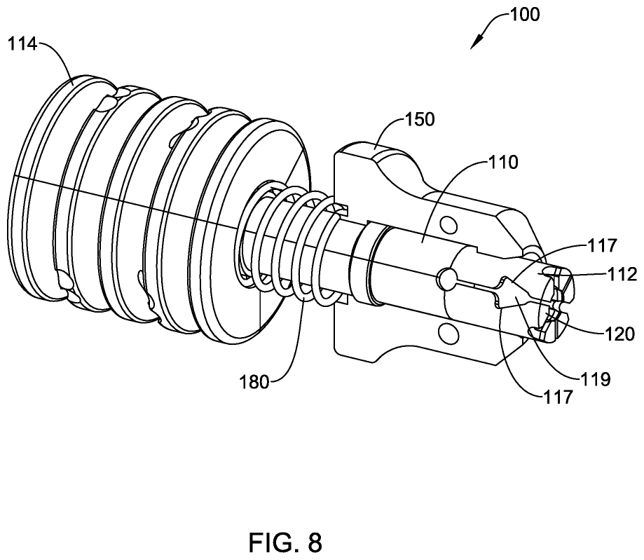

When the plurality of arms 110 is positioned adjacent one another with side edges abutting and inside the lock ring 150, as shown in FIGS. 7 and 8, the side edges of each arm 110 may abut side edges of adjacent arms 110 along the length of each shaft 116 in all regions except the engagement region 112 of each arm 110, as shown in FIG. 7. In FIGS. 7 and 8, half of the lock ring 150 has been removed to show details of the inner structures. The cut-out regions 117 in adjacent arms 110 form the opening 119 when the adjacent arms 110 are in the straight configuration, as shown in FIG. 8. The opening 119 may be configured to receive a pin on the stent holder and a stent loop on the stent portion of the heart valve. In some examples, the opening 119 may have a chamfered lead-in 120 to aid in guiding the stent loop into the opening.

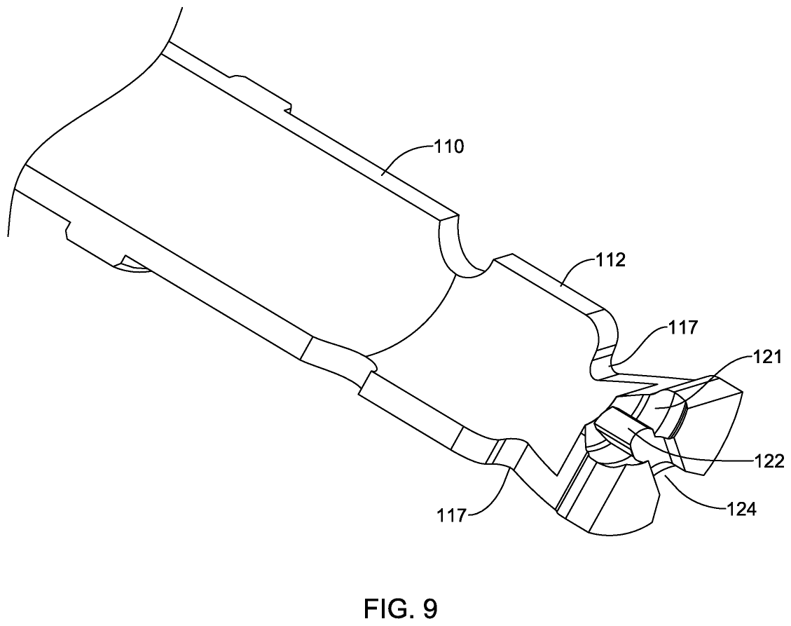

FIG. 9 shows an enlargement of the engagement region 112 of one arm 110, illustrating the cut-out regions 117. In some examples, a holding region 121 is formed on the inner surface of the end of the arms 110. The holding region 121 may be shaped to match or mate with the shape of the stent holder. The holding region 121 may include an inner protrusion 122 configured to engage a slot on the stent holder, to prevent rotation of the stent holder relative to the alignment tool 100. The engagement region 112 may have a longitudinal slit 124 extending from a free end of the engagement region 112 towards the enlarged portion 114 of the arm 110. The longitudinal slit 124 may allow the engagement region 112 to expand when in the angled configuration. Moving the lock ring 150 into the forward locked position may compress the longitudinal slits 124, reducing the inner diameter of the engagement region 112 of the arms around the stent holder.

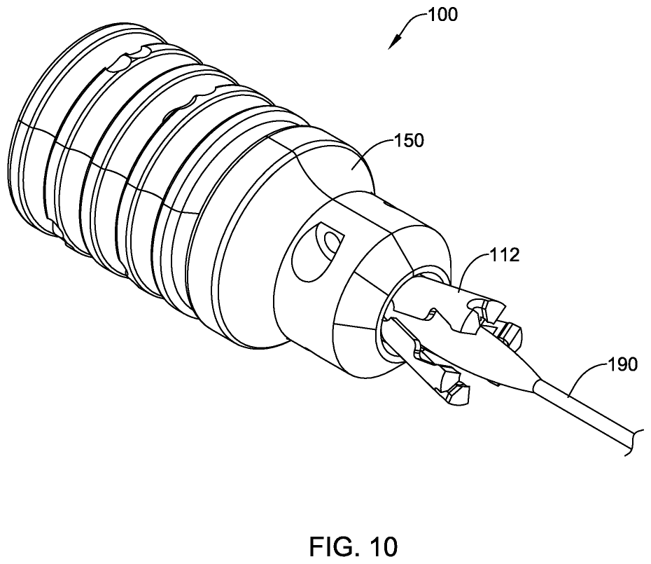

The alignment tool 100 may be used to aid the user in aligning terminal stent loops on a stent or an artificial heart valve with pins on a stent holder. A method of using the alignment tool may include inserting the heart valve or stent having a plurality of terminal end loops into the stent holder having a plurality of pins on which the terminal end loops are to be placed. The alignment tool 100 as discussed above may then be placed over the stent holder 190, as shown in FIG. 10. The alignment tool 100 may be placed over the stent holder 190 with the lock ring 150 in the first retracted position such that the pins on the stent holder are received within the opening 119 in the engagement region 112. The lock ring is released and allowed to move into the locked position, moving the engagement region 112 of each arm into its straight configuration, thereby securing the stent holder 190. The stent may be moved into alignment with the stent loops aligned over the pins. The stent is then compressed onto the stent holder, and the lock ring is moved on the alignment tool to the retracted position to release the stent holder. The alignment tool may then be removed from the stent holder and the stent, the stent loading process is continued.

In some embodiments, one or more components of the alignment tool 100 (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In some embodiments, one or more components of the alignment tool 100 (and variations, systems or components thereof disclosed herein), may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An alignment tool for loading a stent, comprising:
a plurality of arms each having a shaft with a first end including an engagement region and a second opposite end, wherein each engagement region is moveable between a first, angled configuration relative to the shaft, and a second, straight configuration;
a lock ring having a lumen configured to receive the plurality of arms, the lock ring configured to slide over the arms between a first retracted position in which the engagement region of each arm is exposed and allowed to bias into the angled configuration, and a second locked position in which the lock ring extends over at least a portion of the engagement regions and compresses the engagement regions into the straight configuration; and
a spring configured to bias the lock ring in the locked position.

2. The alignment tool of claim 1, wherein each engagement region has a longitudinal slit extending from a free end of the engagement region towards the second end of the arm, the longitudinal slit allowing the engagement region to expand when in the angled configuration.

3. The alignment tool of claim 1, wherein each arm includes a cut-out region in a side surface of the engagement region, wherein the cut-out regions in adjacent arms form an opening when the adjacent arms are in the straight configuration, the opening configured to receive a stent loop.

4. The alignment tool of claim 3, wherein the lock ring includes at least one magnifying lens configured to be positioned over the opening.

5. The alignment tool of claim 1, wherein the lock ring is transparent.

6. The alignment tool of claim 1, wherein each arm has an enlarged portion at the second end of the shaft, wherein the enlarged portion extends radially outward farther than a diameter of the spring.

7. The alignment tool of claim 6, wherein each arm has a raised transverse rib spaced apart from the enlarged portion.

8. The alignment tool of claim 7, wherein the lock ring includes a rear shoulder extending into the lumen, the rear shoulder configured to engage the transverse rib when the lock ring is in the second locked position.

9. The alignment tool of claim 8, wherein the lock ring includes a front shoulder extending into the lumen, the front shoulder configured to slide along the arms and move the engagement regions of the arms from the angled configuration to the straight configuration.

10. The alignment tool of claim 1, wherein each of the plurality of arms is identical.

11. The alignment tool of claim 1, wherein the lock ring has a front end disposed adjacent the engagement region of the arms, and an opposite rear end, wherein the lock ring has an enlarged flared rear end with a diameter larger than a diameter of the front end.

12. The alignment tool of claim 1, wherein each engagement region includes an inner surface shaped to mate with a stent holder.

13. The alignment tool of claim 1, wherein at least at the second end of the plurality of arms, side edges of each arm abut side edges of adjacent arms, thereby defining a channel extending through the plurality of arms.

14. The alignment tool of claim 13, wherein the side edges of each arm abut side edges of adjacent arms along a length of each shaft in all regions but the engagement region of each arm.

15. An alignment tool for loading a stent onto a stent holder, the alignment tool comprising:
a plurality of arms each having a shaft with a first end portion including an engagement region and an enlarged second end portion, wherein each engagement region is moveable between a first, angled configuration relative to the shaft, and a second, straight configuration, wherein side edges of each arm in at least the enlarged second end portion abut side edges of adjacent arms to define a channel;
a lock ring having a lumen configured to receive the shaft of the plurality of arms, the lock ring configured to slide over the arms between a first retracted position in which the engagement region of each arm is exposed and allowed to bias into the angled configuration, and a second locked position in which the lock ring extends over at least a portion of the engagement regions and compresses the engagement regions into the straight configuration; and
a spring configured to bias the lock ring in the locked position.

16. The alignment tool of claim 15, wherein each arm includes a cut-out region in a side surface of the engagement region, wherein the cut-out regions in adjacent arms form an opening when the adjacent arms are in the straight configuration, the opening configured to receive a stent loop.

17. The alignment tool of claim 15, wherein each arm has a raised transverse rib spaced apart from the enlarged portion, the raised transverse rib on all of the arms collectively forming a raised ring.

18. The alignment tool of claim 17, wherein the lock ring includes a rear shoulder extending into the lumen, the rear shoulder configured to engage the raised ring when the lock ring is in the second locked position.

19. The alignment tool of claim 18, wherein the lock ring includes a front shoulder extending into the lumen, the front shoulder configured to slide along the arms and move the engagement regions of the arms from the angled configuration to the straight configuration.

20. A method of loading a stent onto a stent holder using an alignment tool, the method comprising:
inserting the stent having a plurality of terminal end loops into the stent holder having a plurality of pins on which the terminal end loops are to be placed;
placing the alignment tool over the stent holder, the alignment tool having:
a plurality of arms each having a shaft with a first end including an engagement region and a second opposite end, wherein each engagement region is moveable between a first, angled configuration relative to the shaft, and a second, straight configuration, each engagement region having an inner surface shaped to mate with the stent holder, wherein each arm includes a cut-out region in a side surface of the engagement region, wherein the cut-out regions in adjacent arms form an opening configured to receive a stent loop and a pin;
a lock ring having a lumen configured to receive the plurality of arms, the lock ring configured to slide over the arms between a first retracted position in which the engagement region of each arm is exposed and allowed to bias into the angled configuration forming a loading zone for receiving the stent holder, and a second locked position in which the lock ring extends over at least a portion of the engagement regions and compresses the engagement regions into the straight configuration to secure the stent holder; and
a spring configured to bias the lock ring in the locked position;
wherein the alignment tool is placed over the stent holder with the lock ring in the first retracted position such that the pins on the stent holder are received within the opening in the engagement region;
releasing the lock ring and allowing it to move into the locked position, moving the engagement region of each arm into its straight configuration;
advancing the stent and moving one stent loop over each pin;
compressing the stent onto the stent holder;
moving the lock ring on the alignment tool to the retracted position to release the stent holder; and
removing the alignment tool from the stent holder and the stent.
